(19) United States
(12) Patent Application Publication
(10) Pub. No.: US 2011/0071430 A1
(43) Pub. Date: Mar. 24, 2011

(54) Biopsy Device and Method Thereof

(75) Inventors: Woei-Chyn CHU, Taipei (TW); Wen-Cheng CHU, Taipei (TW)

(73) Assignee: NATIONAL YANG MING UNIVERSITY, Taipei (TW)

(21) Appl. No.: 12/630,631

(22) Filed: Dec. 3, 2009

(30) Foreign Application Priority Data
Sep. 18, 2009 (TW) 098131506

(51) Int. Cl. A61B 10/00

(52) U.S. Cl. 600/567

(57) ABSTRACT

A biopsy device includes an acquiring element and a fixation element. The acquiring element has a drill, a sleeve and a handle. The sleeve has first screw threads formed on an outer periphery thereon and is covered on a part of one end of drill. The fixation element has a stylet and a trocar. The stylet has a through hole, and second screw threads are formed on an inner periphery of the through hole. The second screw threads of the through hole are engaged with the first screw threads of the sleeve. The trocar is connected with the through hole for receiving the drill. When the handle is rotated by a force, the acquiring element is moved in the through hole and the torcar relative to the fixation element.

### FIELD OF THE INVENTION

The present invention is related to a biopsy device, and more particularly to a biopsy device having a threaded structure.

### BACKGROUND OF THE INVENTION

Clinically, fine needle biopsy is a technique of medical laboratory test extensively adopted. Live tissue sample is obtained from a human body through a metal probe puncture to perform a pathological analysis and examination further serving to diagnose and treat a disease.

Currently, the fine needle biopsy incorporates different imaging instrument clinically to assist operation. Given a human section image acquired from the imaging instrument for ascertaining relative positions of the probe and a target area, a live tissue sample can be acquired without intruding important blood vessels and organs.

With reference to FIG. 1 illustrating an exploded view of a conventional biopsy device, the conventional biopsy device includes an acquiring element (110) and a fixation element (120). The acquiring element (110) has a drill (111) and a handle (112). The fixation element (120) has a stylet (122) and a trocar (121).

With reference to FIG. 2 illustrating an assembly view of the conventional biopsy device, the stylet (122) has a through hole for partially receiving the drill (111). The trocar (121) is a hollow tube and communicates with the through hole for receiving the drill (111).

The trocar (121) of the fixation element (120) is adopted to pierce through a live body. For example, when choosing cortex as a target tissue sample, the trocar (121) is remained at a position of cortex which is guided through images on the imaging instrument. The stylet (122) is drawn out and replaced by the drill (111), and the drill (111) is received by the trocar (121). At last, the handle (112) is employed to control the drill (111) to acquire the target tissue sample.

The drawback of the conventional biopsy device lies in that actual depth of the drill (111) entering into the trocar (121) is uncertain because of the artifacts on images. The uncertain depth of the drill (111) will easily puts patients in jeopardy. The risk of patients arising from such uncertainty is even more serious when the affected area is in the vicinity of critical nerves and blood vessels.

### SUMMARY OF THE INVENTION

In view of the foregoing shortcoming, the main objective of the present invention is to provide a biopsy device which precisely positions a drill thereof inside a biopsy tissue and aid a surgeon to perform fine needle biopsy positioned by images.

To achieve the foregoing objective, the biopsy device has an acquiring element and a fixation element.

The acquiring element has a drill, a sleeve and a handle. The sleeve takes the form a hollow tube, and has first screw threads on an outer periphery and covered on a part of one end of the stylet.

The fixation element has a stylet and a trocar. The stylet has a through hole, and second screw threads formed on an inner periphery of one end of the through hole and engaged with the first screw threads. The trocar takes the form of a hollow tube and is connected with the through hole for receiving the drill.

The acquiring element is moved relative to the fixation element through the through hole. The acquiring element is tightly fitted with the fixation element through the engagement of the first thread screws on the sleeve and the second screw threads on the fixation element. Besides, the portion of the drill outside the sleeve is also within the trocar. Therefore, when the handle of the acquiring element is rotated by a force, the acquiring element is moved in the through hole and the trocar relative to the fixation element.

The foregoing and other features and advantages of the present invention will be more clearly understood through the following descriptions with reference to the drawing.

### BRIEF DESCRIPTION OF THE DRAWINGS

FIG. 1 is an exploded view of a conventional biopsy device;

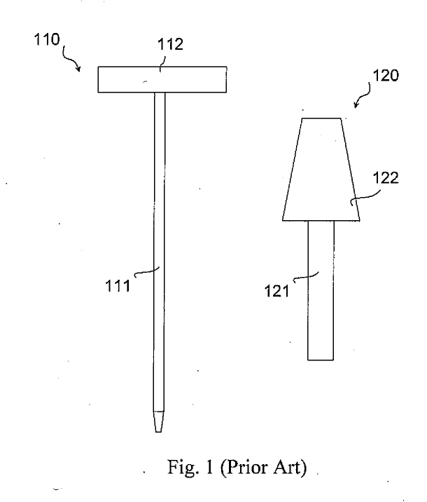

FIG. 2 is an assembly view of the conventional biopsy device;

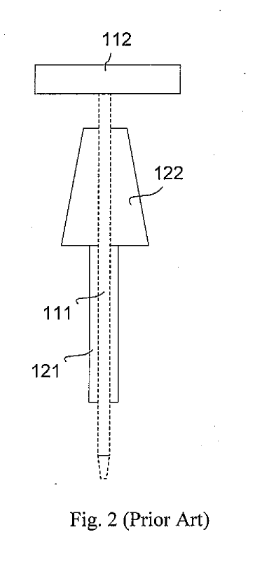

FIG. 3 is a schematic view of a first preferred embodiment of an acquiring element of a biopsy device in accordance with embodiments of this invention;

FIG. 4 is a schematic view of a combinational sleeve of the biopsy device in accordance with embodiments of this invention;

FIG. 5 is a schematic view of a first preferred embodiment of a fixation element of the biopsy device in accordance with embodiments of this invention;

FIG. 6 is a schematic view of the combined acquiring element and the fixation element of the biopsy device in accordance with embodiments of this invention;

FIG. 7 is a schematic view of a drill of the biopsy device in accordance with embodiments of this invention;

FIG. 8 is a schematic view of a second preferred embodiment of the acquiring element of the biopsy device in accordance with embodiments of this invention;

FIG. 9 is a schematic view of a second preferred embodiment of the fixation element of the biopsy device in accordance with embodiments of this invention;

FIG. 10 is a flow diagram of using the biopsy device to acquire biopsy tissue in accordance with embodiments of this invention; and

FIG. 11 is a schematic view about using the biopsy device of the present invention to acquire biopsy tissue.

### DETAILED DESCRIPTION OF THE INVENTION

The biopsy device of the present invention has an acquiring element (200) and a fixation element (300).

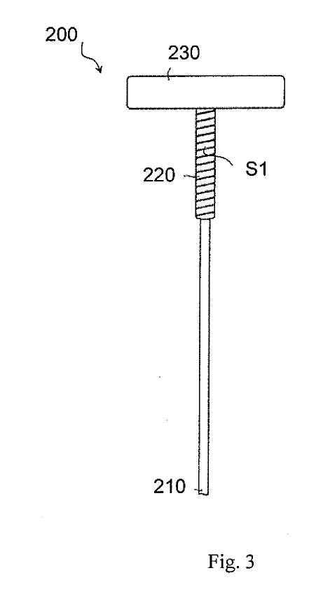

With reference to FIG. 3 illustrating a schematic view of a first preferred embodiment of an acquiring element of a biopsy device in accordance with the present invention, the acquiring element (200) has a drill (210), a sleeve (220) and a handle (230). The sleeve (220) taking the form of a hollow tube (221) has first screw threads S1 formed on an outer wall thereof and is partially sleeved on one end of the drill (210). The sleeve (220) can be also combined with a compression lock structure.

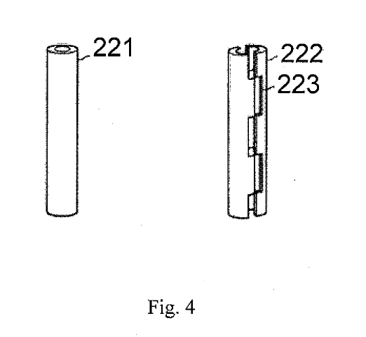

With reference to FIG. 4 illustrating a combinational sleeve of the biopsy device in accordance with the present invention, an assembly member (222) is combined with a compression lock member (223) to form a combinational sleeve (220). The handle (230) abuts one end of the sleeve (220).

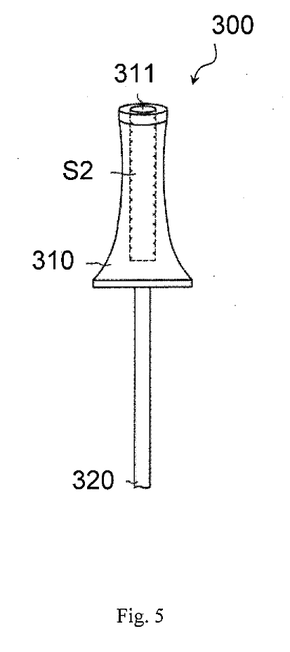

With reference to FIG. 5 illustrating a first embodiment of the fixation element in accordance with the present invention, the fixation element(300) has a stylet (310) and a trocar (320). The stylet (310) has a through hole (311). Second screw threads S2 are formed on an inner periphery of one end of the through hole (311). The second screw threads S2 of the through hole (311) are engaged with the first screw threads S1 of the sleeve (220). The trocar (320) also takes a tubular form and is connected with the through hole (311) for receiving the drill (210).

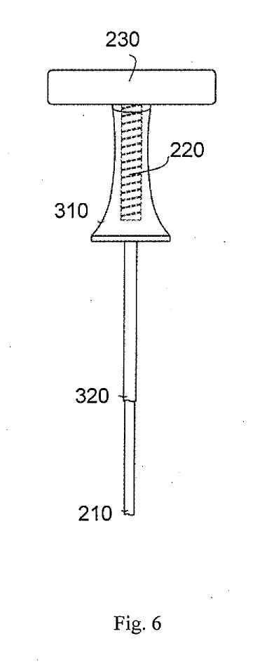

With reference to FIG. 6 illustrating an acquiring element combined with a fixation element of biopsy device in accordance with the present invention, the acquiring element (200) is combined with the fixation element (300) through the through hole (311). The first screw threads S1 of the sleeve (220) of the acquiring element (200) are engaged with the second screw threads S2 on the stylet (310) of the fixation element (300), and the portion of the drill (210) outside the sleeve (220) is also located within the trocar (320). When the handle (230) of the acquiring element (200) is rotated by a force, the acquiring element (200) is moved in the through hole (311) and the trocar (320) relative to the fixation element (300).

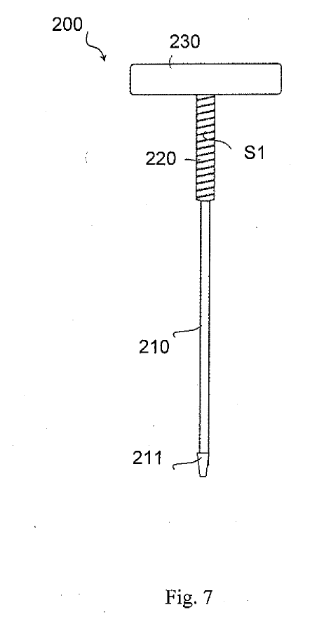

With reference to FIG. 7 illustrating a drill of the biopsy in accordance with the present invention, the drill (210) has a drill bit (211) on a front end thereof for acquiring a target tissue. A barb is formed on an end portion inside the drill bit (211) to prevent the acquired target tissue from dropping when the drill (211) is pulled out.

The diameter of the through hole (311) of the stylet (310) of the first embodiment in accordance with the present invention ranges from 5 mm to 10 mm. Preferably, the diameter is 7 mm. The length of the through hole (311) ranges from 30 mm to 50 mm. Preferably, the length is 40 mm.

The inner diameter of the trocar (320) of the stylet (310) of the first embodiment in accordance with the present invention ranges from 2 mm to 8 mm. Preferably, the inner diameter is 3.4 mm. The outer diameter of the trocar (320) ranges from 3 mm to 9 mm. Preferably, the outer diameter is 6.8 mm. The length of the trocar (320) ranges from 30 mm to 50 mm. Preferably, the length is 40 mm.

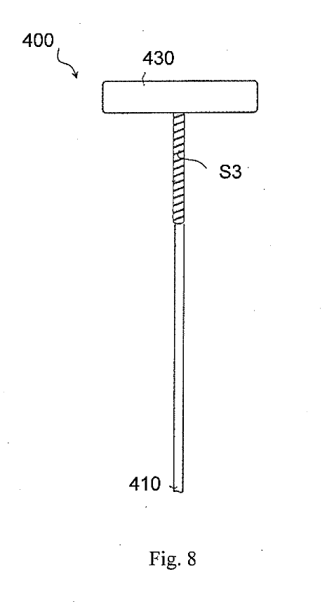

With reference to FIG. 8 illustrating a second embodiment of an acquiring element of the biopsy device in accordance with the present invention, the acquiring element (400) has a drill (410) and a handle (430). The difference between the first and second embodiments of the acquiring element lies in that the acquiring element (400) in the second embodiment has no sleeve (220), the drill (410) is connected with the handle (430), and one end of the drill (410) abutting the handle (430) has third screw threads S3 on an outer periphery thereof.

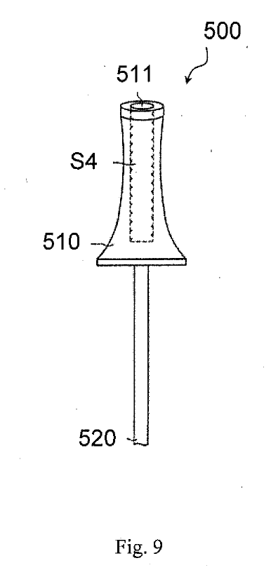

With reference to FIG. 9 illustrating a second embodiment of the fixation element of the biopsy device in accordance with the present invention, the fixation element (500) collaborated with the acquiring element (400) resembles the fixation element (300) in the first embodiment. The difference between the first and second embodiments of the fixation element (500) lies in that the acquiring element (400) is directly connected with the fixation element (500) without going through the sleeve (220). The fixation element (500) has a stylet (510) and a trocar (520). The stylet (510) has a through hole (511), and fourth screw threads S4 are formed on an inner periphery of the through hole (511). The fourth screw threads S4 of the through hole (511) are engaged with the third screw threads S3 of the drill (410). The trocar (520) is also hollow and tubular and is connected with the through hole (511) to receive the drill (410).

The acquiring element (400) is combined with the fixation element (500) through the through hole (511). The third screw threads S3 on the acquiring element (400) are tightly engaged with the fourth screw threads S4 on the stylet (510), and the portion of the drill (410) located outside the through hole (511) is also located inside the trocar (520). When the handle (430) is rotated by a force, the acquiring element (400) is moved in the through hole (511) and the trocar (520) relative to the fixation element (500).

Besides, the handle (230, 430) of the present invention are integrally formed with the drill (210, 410) and may be assembled as well. The acquiring element (200, 400) and the fixation element (300, 500) are precisely positioned by helical motion. Medically, biopsy devices need to collaborate with medical imaging instrument for aiding to ascertain the positions of target biopsy tissues. However, artifacts are generated on images as a result of the characteristics of the materials of biopsy devices, and artifacts affect to correctly position target biopsy tissues. Therefore, using biopsy devices with accurate positioning can lower the danger arising from inaccurate positioning caused by artifacts.

The biopsy device of the present invention employs a drill (210, 410) to acquire target biopsy tissues inside human body. As the length and the width of the drill (210, 410) on images may differ from actual length and width thereof, biopsy devices having accurate positioning greatly facilitate determining correct positions of the drill (210, 410) inside human bodies and effectively avoid to intrude critical nerves or blood vessels.

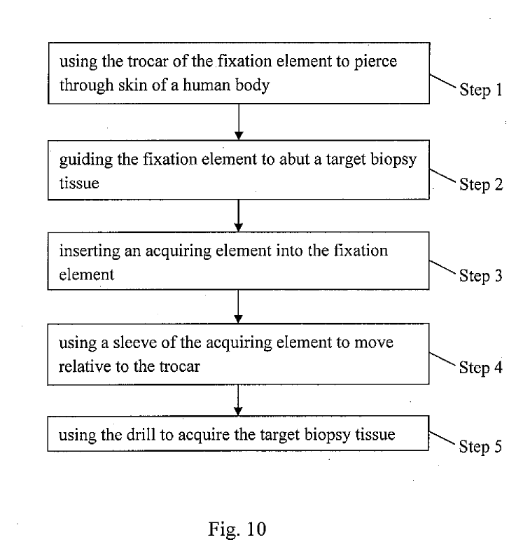

With reference to FIG. 10 illustrating a method for using the biopsy device of the present invention to acquire a biopsy tissue, the method includes steps of:

Step 1: using a trocar of a fixation element to pierce through skin of a human body and remaining a stylet of the fixation element inside the human body;

Step 2: guiding the fixation element to abut a target biopsy tissue through images;

Step 3: inserting an acquiring element into the fixation element through a through hole;

Step 4: using a sleeve of the acquiring element to move relative to the stylet through helical motion of screw threads thereof; the screw threads allow the stylet to move a fixed distance after turning the sleeve one round; hence, it is affirmative to accurately confirm the position of the drill in collaboration with the image guidance; and

Step 5: using the drill to acquire the target biopsy tissue.

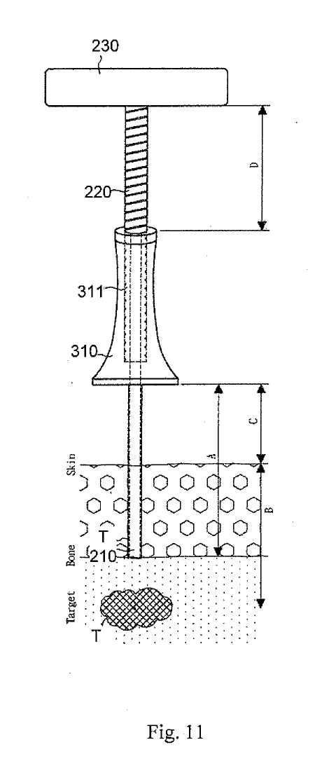

With reference to FIG. 11 illustrating a schematic view about using the biopsy device of the present invention to acquire a target tissue, a distance B from a center T of a target area of a biopsy tissue to skin on an image is measured. The length C of the trocar exposed beyoned the skin is observed by user's naked eyes. Since the length A of the trocar is fixed, the distance of the trocar to the target tissue is B+C−A. As the distance of the acquiring element moved with respect to the stylet is fixed after each round that the sleeve is turned, in other words, the distance B+C−A can be converted into the corresponding number of rounds for the sleeve to turn. The target tissue can thus be correctly acquired.

The biopsy device and method of the present invention let the acquiring element and the fixation element be tightly attached by the structure of screw threads. Given the screw threads with specific pitch, the acquiring element is rotated so that the drill is pierced into a target area of a biopsy tissue and is accurately positioned by convenient calculation with a fixed distance.

While the invention has been described in terms of what is presently considered to be the most practical and preferred embodiments, it is to be understood that the invention needs not be limited to the disclosed embodiments. On the contrary, it is intended to cover various modifications and similar arrangements included within the spirit and scope of the appended claims, which are to be accorded with the broadest interpretation so as to encompass all such modifications and similar structures.

What is claimed is:

1. A biopsy device, comprising:
an acquiring element having
a drill; and
a sleeve taking the form a hollow tube and having first screw threads covered on a part of one end of the stylet;
a fixation element having
a stylet having a through hole, and second screw threads formed on an inner periphery of one end of the through hole and engaged with the first screw threads; and
a trocar taking the form of a hollow tube and connected with the through hole for receiving the drill;
wherein the acquiring element is moved relative to the fixation element when the acquiring element is rotated by a force.

2. The biopsy device as claimed in claim 1, wherein the acquiring element further comprises a handle abutting one end of the sleeve.

3. The biopsy device as claimed in claim 1, wherein the drill has a drill bit on a front end thereof.

4. The biopsy device as claimed in claim 3, wherein a barb is formed on an end portion inside the drill bit.

5. The biopsy device as claimed in claim 2, wherein the handle and the drill are integrally formed.

6. The biopsy device as claimed in claim 1, wherein the sleeve is combined by a compression lock structure.

7. The biopsy device as claimed in claim 1, wherein an inner diameter of the through hole is in a range of 5 mm˜10 mm.

8. The biopsy device as claimed in claim 1, wherein a length of the through hole is in a range of 30 mm˜50 mm.

9. A biopsy device, comprising:
an acquiring element having
a drill comprising first screw threads formed on an outer periphery of one end of the drill;
a fixation element comprising:
a stylet having a through hole, and second screw threads formed on an inner periphery of one end of the through hole and engaged with the first screw threads; and
a trocar taking the form of a hollow tube and connected with the through hole for receiving the drill;
wherein the acquiring element is moved relative to the fixation element when the acquiring element is rotated by a force.

10. A biopsy method using a biopsy device having an acquiring element and a fixation element, the acquiring element having a drill and a sleeve, and the fixation element having a stylet and a trocar, comprising steps of:
using the trocar of the fixation element to pierce through skin of a human body;
guiding the fixation element to abut a target biopsy tissue;
inserting an acquiring element into the fixation element;
using a sleeve of the acquiring element to move relative to the trocar; and
using the drill to acquire the target biopsy tissue.

11. The biopsy method as claimed in claim 10, wherein the sleeve and the stylet are relatively moved by turning screw threads respectively formed thereon.

12. The biopsy method as claimed in claim 10, wherein a drill bit of the drill is adopted to acquire the target biopsy tissue.

13. The biopsy method as claimed in claim 10, wherein the drill has a barb to prevent the acquired target biopsy tissue from dropping.
